(19) United States
(12) Patent Application Publication
(10) Pub. No.: US 2005/0226882 A1
(43) Pub. Date: Oct. 13, 2005

(54) Method and multicomponent conjugates for treating cancer

(76) Inventor: Essam T. Awdalla, Raleigh, NC (US)

Correspondence Address: Essam T. Awdalla;Apt. F, 2309 Myron Drive, Raleigh, NC, 27607, US

(21) Appl. No.: 11/049,118

(22) Filed: Feb. 2, 2005

Related U.S. Application Data
(63) Continuation-in-part of application No. 10/820,569, filed on Apr. 8, 2004.
(63) Continuation-in-part of application No. 11/020,855, filed on Dec. 23, 2004.

(51) Int. Cl. A61K 39/395; C07K 16/46

(52) U.S. Cl. 424/155.1; 530/391.1; 424/178.1

(57) ABSTRACT

The present invention provides a method for treating cancer in a mammal and to using multicomponent conjugates of formula: A--B--C, where “B” may be present or absent, “A” comprises at least one specific anti-tumor binding protein recognizing a tumor associated receptor, “B” comprises at least one molecule to which “A” and “C” bind, “C” comprises at least one specific anti-platelet binding protein recognizing a receptor present on the outer surface of a blood platelet. The multicomponent conjugates provide the exquisite binding specificity, which enables attaching the blood platelets either to the outer surface of the cancer cells, or to the outer surface of a component of the tumor associated vasculature or stroma, and thus, stimulating the formation of blood clots within the blood vessels around and in-between cancer cells, leading to their destruction.

### CROSS-REFERENCE TO RELATED APPLICATION(S)

This non-provisional utility patent application claims the benefit of one or more prior filed co-pending non-provisional applications; the present application is a continuation-in-part of U.S. patent application Ser. No. 11/020,855, filed Dec. 23, 2004, which is incorporated herein by reference in its entirety.

### FIELD OF THE INVENTION

The present invention relates generally to a method for treating cancer in a mammal and to multicomponent conjugates useful in such treatment, and more specifically to a method and multicomponent conjugates which are effective in shrinking the size of established tumors, or clearing tumors entirely from the mammalian body.

### BACKGROUND OF THE INVENTION

Cancer is one of the most serious diseases threatening human and animal health and life. Treatment of cancer in the early stages typically comprises local treatment such as surgery and/or radiotherapy. More advanced diseases are usually treated by combining local treatment with chemotherapy. Although current chemotherapeutic agents are effective against cancers and tumor cells, the use of combined treatment with all three modalities i.e., surgery, radiotherapy, and chemotherapy, have not shown to be effective against all cancer and tumor cells, due to the wide heterogeneity of cancer cells regarding their metabolism, enzyme composition, growth rate and gene errors, with some of the cancer cells being usually resistant to each of the used treatment modalities. The resistant cells survive, seed, and continue to grow in the living host, with subsequent treatments being less effective at killing the cancer cells.

Also, as the tumor grows, in order to sustain itself, it must develop its own blood supply. This blood supply, however, is much different from the blood supply to normal tissues. The blood vessels formed in tumors are typically highly irregular and tortuous. They may have arterio-venous shunts and blind ends, and lack smooth muscle or nerves and have incomplete endothelial linings and basement membranes. This leads to low overall levels of oxygen in most tumors. Many tumors have areas of extreme hypoxia. (Brown, J. M. “Exploiting the hypoxic cancer cell: mechanisms and therapeutic strategies.” Molecular Medicine Today, April 2000 (Vol. 6)). Such hypoxic areas are known to be refractory towards many of the currently available treatments for solid tumor cancers, including radiation therapy and chemotherapy.

Several unconventional approaches for treating cancer are being proposed continuously. One approach related to the present invention, but not relied upon, is disclosed in U.S. Pat. Nos. 5,877,289, 6,004,555 and 6,093,399 by Thorpe et al, which provide a method for in vivo coagulation of tumor vasculature, causing tumor regression, through the site-specific delivery of a coagulant, using a binding ligand including a binding region operatively associated with or linked to a “coagulating agent”. However, as this method entails intravenous injection of a ligand including or associated with a “coagulating agent”, so it may result in the formation of micro-emboli within the vasculature of other vital body organs.

Accordingly, there exists a need for a method of treating solid tumor cancers having resistant cells and/or hypoxic regions, without the risk of forming micro-emboli within the vasculature of vital body organs.

Prior art documents include components related to the field of the present invention but lack an integrated, combined solution that is provided by the present invention, including the following:

Monoclonal antibodies and their fragments, which may be derived from any species (including humans) or may be formed as chimeric proteins which employ sequences from more than one species, using conventional techniques, such as hybridoma synthesis, recombinant DNA techniques and protein synthesis. See, generally, Kohler and Milstein, Nature, 256: 495-97, 1975; and Eur. J. Immunol., 6: 511-19, 1976; both of which are incorporated herein by reference;

human, or humanized, monoclonal antibodies recognizing surface antigens of cancer cells. Non limiting examples are described by Hosokawa, et al. in U.S. Pat. No. 6,787,153, by Taniguchi, et al. in U.S. Pat. No. 4,800,155, by Abe, et al. in U.S. Pat. No. 5,024,946, by Hagiwara, et al. in U.S. Pat. No. 5,093,261, and by Anderson, et al. in U.S. Pat. No. 6,753,420 and U.S. Pat. No. 6,417,337; all of which are incorporated herein by reference;

antibodies recognizing tumor associated antigens. Non limiting examples includes antibodies targeting tumor vasculature (Duijvestijn et al., J. Immunol., 138(3):713-719, 1987; Hagemeier et al., Int. J. Cancer, 38:481-488, 1986; Bruland et al., Int. J. Cancer, 38:27-31, 1986; Murray et al., Radiotherapy and Oncology, 16:221-234, 1989; and Schlingemann et al., Laboratory Investigation, 52(1):71-76, 1985), and antibodies targeting tenascin, a large molecular weight extracellular glycoprotein expressed in the stroma of various benign and malignant tumors (Shrestha et. al., Eur. J. Cancer B. Oral. Oncol., 30B(6):393-9, 1994; and Tuominen and Kallioinen, J. Cutan. Pathol. 21(5):424-9, 1994.), all of which are incorporated herein by reference;

anti-platelet antibodies described by Gralnick in U.S. Pat. No. 5,366,865, which is incorporated herein by reference;

and the use of streptavidin, avidin, and biotin molecules to conjugate molecules to one another, to form biotinylated protein molecules, biotinylated protein-avidin or avidin like complexes, or multicomponent conjugates, both in vitro and in vivo, is well known in the Art. See, generally, P. Webber et al., “Science, vol. 243, pp. 85-88, Jan. 6, 1989”, M. Wilchek et al, “Analytical Biochemistry, vol. 171 pp. 1-32, 1988”, Otto C. Boerman et al., “Pretargeted Radioimmunotherapy of Cancer: Progress Step by Step”, Journal of Nuclear Medicine Vol. 44 No. 3 400-411, Bayer et al., “Trends in Biochemical Science, 3, N257, November 1978”, and Paganelli G, Riva P, Deleide G, et al. “Int J Cancer Suppl. 1988; 2: 121-125”; all of which are incorporated herein by reference.

However, non of these references suggest that anti-cancer antibodies, or antibodies recognizing tumor associated antigens, could be linked to anti-platelet antibodies, which enables attaching the blood platelets to the outer surface of cancer cells, or to the outer surface of tumor associated vasculature or stroma, and thus stimulating the selective formation and immobilization of blood clots within the blood vessels around and in-between the cancer cells, leading to their destruction.

### SUMMARY OF THE INVENTION

The present invention is directed to and provides, in one aspect of the invention, a method for treating cancer by stimulating the formation of blood clots within the blood vessels around and in-between the cancer cells, leading to their destruction. The present invention is further directed, in another aspect of the invention, to multicomponent conjugates or fusion proteins, used for attaching the blood platelets to cancer cells, or to tumor associated vasculature or stroma, and thus stimulating the formation of blood clots within the blood vessels around and in-between the cancer cells.

As used herein, the term “anti-tumor binding protein recognizing a tumor associated antigen or receptor” refers to and includes any binding protein recognizing an antigen or a receptor on the outer surface of a cancer cell, or on the outer surface of a component of tumor-associated vasculature or stroma; the term “anti-tumor antibody” refers to and includes any antibody that recognizes an antigen on the outer surface of a cancer cell, or on the outer surface of a component of tumor-associated vasculature or stroma; and the term “a ligand recognizing a tumor associated receptor” refers to and includes any ligand that recognizes a receptor on the outer surface of a cancer cell, or on the outer surface of a component of tumor-associated vasculature or stroma.

Accordingly, the cancer cells, or the tumor associated vasculature or stroma, are tagged with a number of multicomponent conjugates, each having the formula A--B--C, where “B” may be present or absent, “A” comprises at least one specific anti-tumor binding protein recognizing a tumor associated antigen or receptor, such as an anti-tumor antibody or an anti-tumor antibody binding fragment, or a ligand recognizing a tumor associated receptor, “B” comprises at least one molecule to which “A” and “C” bind, such as a biotin-avidin-biotin complex, “C” comprises at least one specific anti-platelet binding protein such as an anti-platelet antibody or an anti-platelet antibody binding fragment, or a ligand recognizing a receptor present on the outer surface of a blood platelet. The multicomponent conjugates will attach the blood platelets to the cancer cells, or to the tumor associated vasculature or stroma.

As the life span of blood platelets is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood clot to the outer surface of the activated blood platelets. And thus, the formed blood clot will be indirectly attached to the cancer cells.

The formed blood clot will first occlude the blood vessels in-between the cancer cells, followed by extension of the clot to the main tumor feeding and draining blood vessels. The extension of the clot to the main feeding vessels is favored by the stagnant current resulting from the blood clot formed within the blood vessels in-between the cancer cells.

The formed blood clot will cut off the blood supply to the cancer cells, leading to their destruction. However, as only the relatively centrally located cancer cells (within a solid tumor) depend for their nutrition on the feeding blood vessels, while the relatively peripherally located cancer cells depend on nearby tissue fluids for their nutrition, so it is expected that peripherally located cancer cells will survive, unless the feeding blood vessels of the nearby tissue are also occluded, which is not expected to always happen.

To safeguard against this, the method provided in the present invention is preferably used in combination with at least one other treatment modality known to specifically target peripherally located cancer cells, e.g. Chemotherapy, Conventional radiotherapy, or by using radio-labeled monoclonal antibodies. When Chemotherapy or Conventional radiotherapy are used, they are administered before, with, or after the method provided in the present invention. When radio-labeled monoclonal antibodies are used, they are preferably administered prior to the method provided in the present invention, as in this case the formed blood clot will trap the radio-labeled monoclonal antibodies within the occluded blood vessels in the relatively central part of the tumor, leading to prolonged exposure of the surviving peripherally located cancer cells to the ionizing radiation, which favors their complete destruction. Also, in this case, the prolonged exposure of the nearby tissues to the ionizing radiation will favor the destruction of the endothelial lining of their feeding blood vessels, leading to their obliteration, which further favors the destruction of the peripherally located cancer cells.

The multicomponent conjugates are either prepared in vitro and administered to the mammal, or the components forming the multicomponent conjugates are administered to the mammal individually, or after linking some of them to one another, so that the multicomponent conjugates are assembled in vivo within the body of the mammal, using one of the pretargeting strategies described herein after.

These and other aspects of the present invention will become apparent to those skilled in the art after a reading of the following description of the preferred embodiment when considered with the drawings.

### BREIF DESCRIPTION OF THE DRAWINGS

The description of the features of the present invention will be more fully appreciated by reference to the following detailed description of the exemplary embodiments in accordance with the accompanying drawings, wherein:

FIG. 1 is a schematic representation of a 3 step method, showing the use of the multicomponent conjugates provided in the present invention for treating a mammal suffering from cancer.

FIG. 2 is a schematic representation of a 2 step method, showing the use of the multicomponent conjugates provided in the present invention for treating a mammal suffering from cancer.

FIG. 3 is a schematic representation of another 3 step method, showing the use of the multicomponent conjugates provided in the present invention for treating a mammal suffering from cancer.

FIG. 4 is a schematic representation of another 2 step method, showing the use of the multicomponent conjugates provided in the present invention for treating a mammal suffering from cancer.

FIG. 5 is a schematic representation another 2 step method, showing the use of the multicomponent conjugates provided in the present invention for treating a mammal suffering from cancer.

FIG. 6 is a schematic representation of another 3 step method, showing the use of the multicomponent conjugates provided in the present invention for treating a mammal suffering from cancer.

FIG. 7 is a schematic representation of a 4 step method, showing the use of the multicomponent conjugates provided in the present invention for treating a mammal suffering from cancer.

### DETAILED DESCRIPTION OF THE INVENTION

The present invention is directed to and provides, in one aspect of the invention, a method for treating cancer by stimulating the formation of blood clots within the blood vessels around and in-between the cancer cells, leading to their destruction. The present invention is further directed, in another aspect of the invention, to multicomponent conjugates or fusion proteins, used for attaching the blood platelets to cancer cells, or to tumor associated vasculature or stroma, and thus stimulating the formation of blood clots within the blood vessels around and in-between the cancer cells.

As used herein, the term “anti-tumor binding protein recognizing a tumor associated antigen or receptor” refers to and includes any binding protein recognizing an antigen or a receptor on the outer surface of a cancer cell, or on the outer surface of a component of tumor-associated vasculature or stroma; the term “anti-tumor antibody” refers to and includes any antibody that recognizes an antigen on the outer surface of a cancer cell, or on the outer surface of a component of tumor-associated vasculature or stroma; and the term “a ligand recognizing a tumor associated receptor” refers to and includes any ligand that recognizes a receptor on the outer surface of a cancer cell, or on the outer surface of a component of tumor-associated vasculature or stroma.

Accordingly, the cancer cells, or the tumor associated vasculature or stroma, are tagged with a number of multicomponent conjugates, each having the formula A--B--C, where “B” may be present or absent, “A” comprises at least one specific anti-tumor binding protein recognizing a tumor associated antigen or receptor, such as an anti-tumor antibody or an anti-tumor antibody binding fragment, or a ligand recognizing a tumor associated receptor, “B” comprises at least one molecule to which “A” and “C” bind, such as a biotin-avidin-biotin complex, “C” comprises at least one specific anti-platelet binding protein such as an anti-platelet antibody or an anti-platelet antibody binding fragment, or a ligand binding to a receptor present on the outer surface of a blood platelet. The multicomponent conjugates will attach the blood platelets to the cancer cells, or to the tumor associated vasculature or stroma.

As the life span of blood platelets is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood clot to the outer surface of the activated blood platelets. And thus, the formed blood clot will be indirectly attached to the cancer cells.

The formed blood clot will first occlude the blood vessels in-between the cancer cells, followed by extension of the clot to the main tumor feeding and draining blood vessels. The extension of the clot to the main feeding vessels is favored by the stagnant current resulting from the blood clot formed within the blood vessels in-between the cancer cells.

The formed blood clot will cut off the blood supply to the cancer cells, leading to their destruction. However, as only the relatively centrally located cancer cells (within a solid tumor) depend for their nutrition on the feeding blood vessels, while the relatively peripherally located cancer cells depend on nearby tissue fluids for their nutrition, so it is expected that peripherally located cancer cells will survive, unless the feeding blood vessels of the nearby tissue are also occluded, which is not expected to always happen.

To safeguard against this, the method provided in the present invention is preferably used in combination with at least one other treatment modality known to specifically target peripherally located cancer cells, e.g. Chemotherapy, Conventional radiotherapy, or by using radio-labeled monoclonal antibodies. When Chemotherapy or Conventional radiotherapy are used, they are administered before, with, or after the method provided in the present invention. When radio-labeled monoclonal antibodies are used, they are preferably administered prior to the method provided in the present invention, as in this case the formed blood clot will trap the radio-labeled monoclonal antibodies within the occluded blood vessels in the relatively central part of the tumor, leading to prolonged exposure of the surviving peripherally located cancer cells to the ionizing radiation, which favors their complete destruction. Also, in this case, the prolonged exposure of the nearby tissues to the ionizing radiation will favor the destruction of the endothelial lining of their feeding blood vessels, leading to their obliteration, which further favors the destruction of the peripherally located cancer cells.

The multicomponent conjugates are either prepared in vitro and administered to the mammal, or the components forming the multicomponent conjugates are administered to the mammal individually, or after linking some of them to one another, so that the multicomponent conjugates are assembled in vivo within the body of the mammal, using one of the pretargeting strategies described herein after.

The present invention provides a method for treating cancer by stimulating the formation of blood clots within the blood vessels around and in-between cancer cells, leading to their destruction. The present invention also provides multicomponent conjugates or fusion proteins, each having the formula A--B--C, where “B” may be present or absent, “A” comprises at least one specific anti-tumor binding protein recognizing a tumor associated antigen or receptor, such as an anti-tumor antibody or an anti-tumor antibody binding fragment, or a ligand recognizing a tumor associated receptor, “B” comprises at least one molecule to which “A” and “C” bind, such as a biotin-avidin-biotin complex, “C” comprises at least one specific anti-platelet binding protein such as an anti-platelet antibody or an anti-platelet antibody binding fragment, or a ligand recognizing a receptor present on the outer surface of a blood platelet. The multicomponent conjugates will attach the blood platelets to the cancer cells, or to the tumor associated vasculature or stroma, and thus stimulating the formation of blood clots within the blood vessels around and in-between the cancer cells.

It is preferred that the “A” anti-tumor binding protein is an antibody or a binding portion of an antibody, such as a Fab′ fragment or an F(ab′).sub.2 fragment or a single chain Fv fragment. The antibody, or its binding fragment, is chosen so as to create a conjugate that binds specifically to a cancer cell surface antigen, or to an antigen displayed on the outer surface of a component of tumor-associated vasculature or stroma. Non-limiting examples of antigens to which antibodies may be directed includes CEA (carcinoembryonic antigen), A33 (colon cancer), G250 (renal cancer), 3S193 (anti-Lewis Y antibody) effective against epithelial cancers, such as carcinomas, breast, epithelial, colon, and lung cancer, and KM871/KWL871, which is effective against melanoma. Such tumor associated antigens and monoclonal antibodies recognizing them are well known to people experienced in the Art.

Whereas anti-tumor antibodies and binding fragments of antibodies are preferred, other anti-tumor binding proteins can be used. For example, “A” may be a ligand molecule, or a portion of such a molecule, known to be involved in a receptor/ligand interaction. Exemplary of such interactions is that between epidermal growth factor (EGF) receptor and EGF and others are well known, and need not be repeated here.

The “C” anti-platelet binding protein is preferably an anti-platelet antibody or a binding portion of an antibody, such as a Fab′ fragment or an F(ab′).sub.2 fragment or a single chain Fv fragment. The antibody, or its binding fragment, is chosen so as to create a conjugate that binds specifically to a platelet surface antigen, which will not interfere with the ADP or fibrinogen-induced platelet activation and aggregation. A non-limiting example includes the anti-platelet monoclonal antibodies described by Gralnick in U.S. Pat. No. 5,366,865, which only inhibits collagen induced platelet aggregation, and which is incorporated herein by reference.

When “B” is not present in the conjugates, “A” and “C” may be prepared via the use of e.g., nucleic acid coding constructs which encode fusion polypeptides. One may also modify the elements “A” and “C” to connect them chemically. One may add amino acid sequences such as those found in the Jun and Fos oncogenes, which then bind A and C via leucine zipper formation. These and other available alternatives are well known by people skilled in the Art.

When “B” is used, this comprises a molecule or molecules which facilitate the linking of “A” and “C”. In a preferred embodiment, “B” comprises a specific binding pair of molecules, or a complex thereof, such as a complex of avidin or streptavidin or a chemically modified form of streptavidin or avidin, and biotin molecules, wherein the number of the “A” molecules ranges from 1 to 3, and the number of the “C” molecules ranges from 1 to 3, with each of the “A” and “C” molecules being attached to one of the four binding sites of the used streptavidin, avidin, or one of their chemically modified forms through a biotin molecule. In another preferred embodiment, “B” comprises a bispecific antibody with one arm directed against a “Tag” epitope placed at the C terminus of “A”, and the other arm directed against another “Tag” epitope placed at the C terminus of “C”. These and other available alternatives are well known by people skilled in the Art. See, generally, P. Webber et al., “Science, vol. 243, pp. 85-88, Jan. 6, 1989”, M. Wilchek et al, “Analytical Biochemistry, vol. 171 pp. 1-32, 1988”, Bayer et al., “Trends in Biochemical Science, 3, N257, November 1978”, and Paganelli G, Riva P, Deleide G, et al. “Int J Cancer Suppl. 1988; 2: 121-125”; all of which are incorporated herein by reference.

The multicomponent conjugates are either prepared in vitro and administered to the mammal, or the components forming the multicomponent conjugates are administered to the mammal individually, or after linking some of them to one another, so that the multicomponent conjugates are assembled in vivo within the body of the mammal, using one of the pretargeting strategies described herein after.

As used herein, the expression “to attach a component to a tumor” refers to and includes attaching a component to the outer surface of a cancer cell, or to the outer surface of a component of tumor-associated vasculature or stroma.

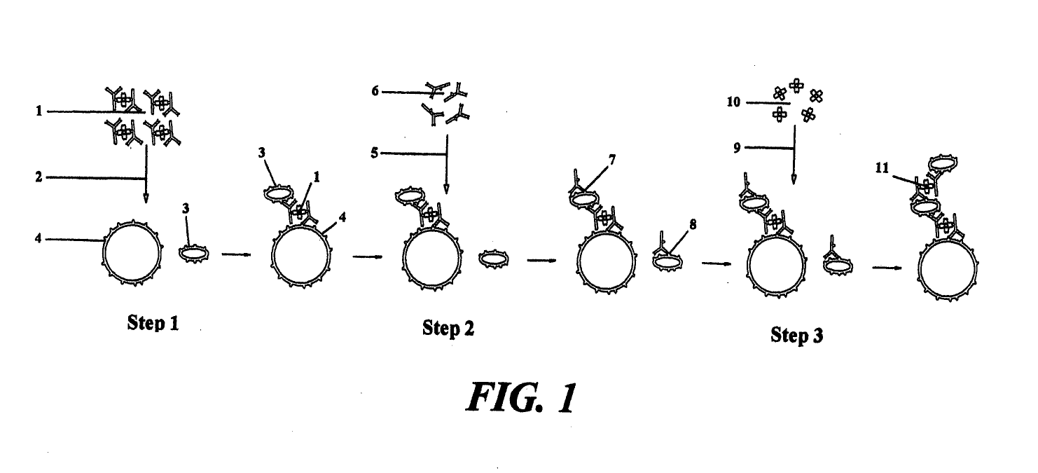

FIG. 1 is a schematic representation of a 3 step method, showing the use of the multicomponent conjugates provided in the present invention for treating a mammal suffering from cancer, by attaching the blood platelets to the tumor. The provided method comprises the steps of:

1) Preparing a number of multicomponent conjugates (1), each including at least one specific anti-tumor binding protein recognizing a tumor associated antigen or receptor, and at least one specific anti-platelet binding protein recognizing a receptor or an antigen present on the outer surface of blood platelets, using one of the before mentioned strategies, followed by administration (2) of the multicomponent conjugates to the mammal preferably through intravenous injection. The administered multicomponent conjugates (1) attach the blood platelets (3) to the tumor (4).

2) Intravenous injection (5) of a number of biotinylated anti-platelet antibodies (6). The administered anti-platelet antibodies will attach themselves to the outer surfaces of the blood platelets already attached to the tumor (7) and to the outer surfaces of a number of platelets freely circulating in the blood (8).

3) Intravenous injection (9) of a number of streptavidin, avidin, or one of their chemically modified molecules (10). The administered avidin or avidin like molecules will attach the freely circulating blood platelets having biotinylated antibodies attached to their outer surfaces to the blood platelets already attached to the tumor through biotin-avidin-biotin or biotin-avidin like-biotin linkages (11).

This 3 step method provides means for accelerating the process of formation of blood clots within the blood vessels around and in-between the cancer cells. However, the administration of the avidin or avidin like molecules (10) in step 3 may be associated with clumping of the freely circulating platelets having biotinylated antibodies attached to their outer surfaces (8), leading to the formation of multiple thrombi within the blood vessels. So, this method is to be used very cautiously in elderly patients and in patients having a tendency to develop infarctions.

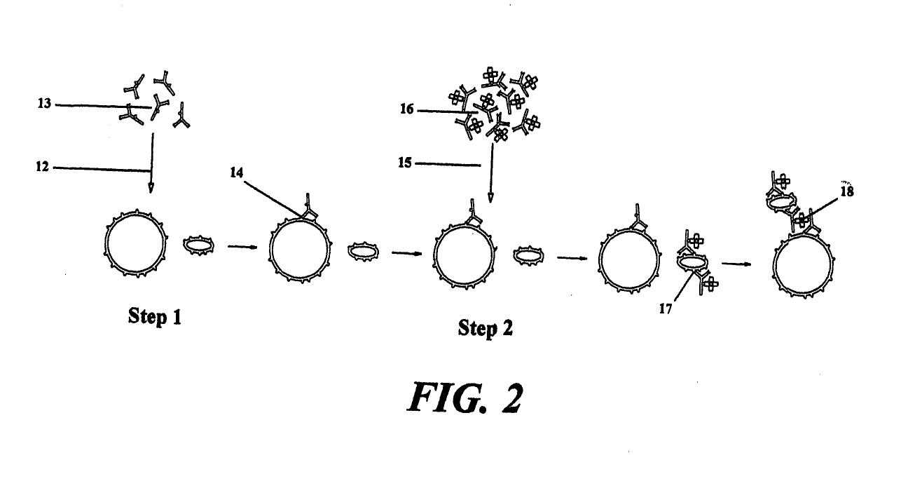

FIG. 2 is a schematic representation of a 2 step method, showing the use of the multicomponent conjugates provided in the present invention for treating a mammal suffering from cancer.

The provided method comprises the steps of:

1) Intravenous injection (12) of a number of biotinylated anti-tumor antibodies (13). The administered anti-tumor antibodies will attach themselves to the tumor (14).

2) Intravenous injection (15) of a number of biotinylated anti-platelet antibodies-avidin or avidin like complexes (16). The administered complexes will attach themselves to the outer surfaces of the blood platelets (17) freely circulating in the blood, followed by attaching the blood platelets to the tumor through the formation of biotin-avidin-biotin or biotin-avidin like-biotin linkages with the biotinylated anti-tumor antibodies already attached to the tumor (18).

This 2 step method provides means for accelerating the rate with which the biotinylated anti-tumor antibodies accumulate within the tumor, and hence increasing the rate with which blood platelets are immobilized within the blood vessels around and in-between the cancer cells, with ultimate blood clot formation. Also, it safes guard against the formation of thrombi within the blood vessels of elderly patients and patients having a tendency to develop infarctions.

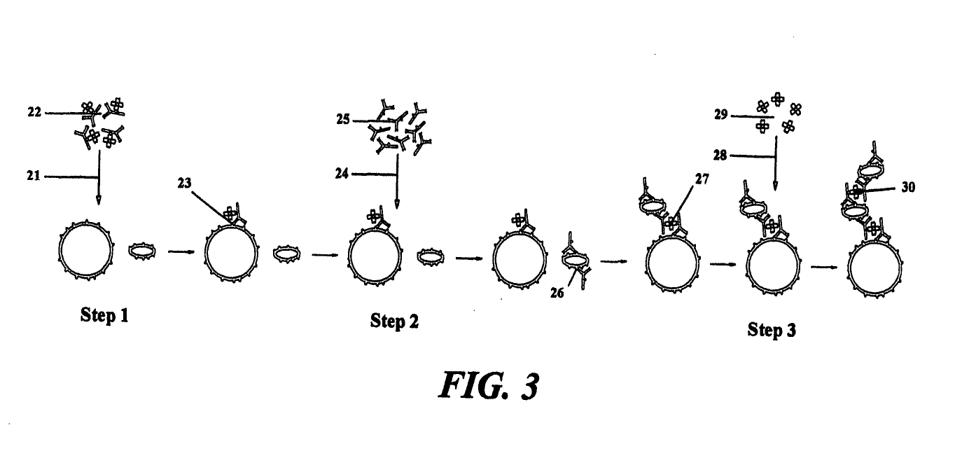

FIG. 3 is a schematic representation of another 3 step method, showing the use of the multicomponent conjugates provided in the present invention for treating a mammal suffering from cancer.

The provided method comprises the steps of:

1) Intravenous injection (21) of a number of biotinylated anti-tumor antibodies-avidin or avidin like complexes (22). The administered complexes will attach themselves to the tumor (23).

2) Intravenous injection (24) of a number of biotinylated anti-platelet antibodies (25). The administered antibodies will attach themselves to the outer surfaces of the blood platelets freely circulating in the blood (26), followed by attaching the blood platelets to the tumor through the formation of biotin-avidin-biotin or biotin-avidin like-biotin linkages (27) with the antibodies already attached to the tumor.

3) Intravenous injection (28) of a number of streptavidin, avidin, or one of their chemically modified molecules (29). The administered avidin or avidin like molecules will attach the extra freely circulating blood platelets having biotinylated antibodies attached to their outer surfaces to the blood platelets already attached to the tumor through biotin-avidin-biotin or biotin-avidin like-biotin linkages (30).

This 3 step method also provides means for accelerating the process of formation of blood clots within the blood vessels around and in-between the cancer cells. However, the administration of the avidin or avidin like molecules (29) in step 3 may be associated with clumping of the freely circulating platelets having biotinylated antibodies attached to their outer surfaces (26), leading to the formation of multiple thrombi within the blood vessels. So, this method is to be used very cautiously in elderly patients and in patients having a tendency to develop infarctions. Optionally, the third step may be omitted, which enables the use of this method in elderly patients and patients having a tendency to develop infarctions.

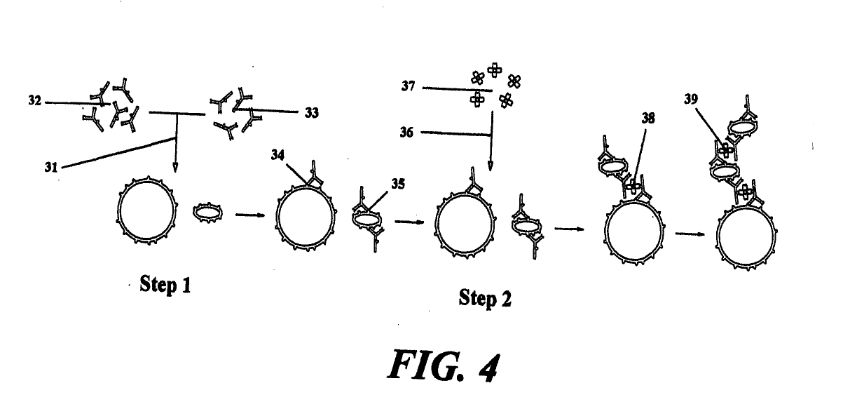

FIG. 4 is a schematic representation of another 2 step method, showing the use of the multicomponent conjugates provided in the present invention for treating a mammal suffering from cancer.

The provided method comprises the steps of:

1) Intravenous injection (31) of a number of biotinylated anti-tumor antibodies (32) and another number of biotinylated anti-platelet antibodies (33), either simultaneously or successively in any order. The administered anti-tumor and anti-platelet antibodies will attach themselves to the outer surfaces of the tumor (34) and the blood platelets (35) respectively.

2) Intravenous injection (36) of a number of streptavidin, avidin, or one of their chemically modified molecules (37). The administered avidin or avidin like molecules will attach the blood platelets to the tumor through biotin-avidin-biotin or biotin-avidin like-biotin linkages (38), followed by attaching the extra freely circulating blood platelets having biotinylated antibodies attached to their outer surfaces to the blood platelets already attached to the tumor, also through biotin-avidin-biotin or biotin-avidin like-biotin linkages (39).

This 2 step method also provides means for accelerating the process of formation of blood clots within the blood vessels around and in-between the cancer cells. However, the administration of the avidin or avidin like molecules (37) in step 2 may be associated with clumping of the freely circulating platelets having biotinylated antibodies attached to their outer surfaces (35), leading to the formation of multiple thrombi within the blood vessels. So, this method is to be used very cautiously in elderly patients and in patients having a tendency to develop infarctions.

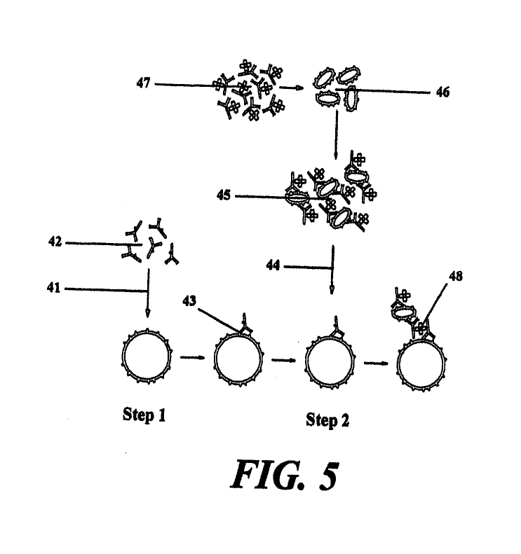

FIG. 5 is a schematic representation another 2 step method, showing the use of the multicomponent conjugates provided in the present invention for treating a mammal suffering from cancer.

The provided method comprises the steps of:

1) Intravenous injection (41) of a number of biotinylated anti-tumor antibodies (42). The administered anti-tumor antibodies will attach themselves to the tumor (43).

2) Intravenous injection (44) of a number of, in vitro prepared, blood platelets having biotinylated anti-platelet antibodies-avidin or avidin like complexes attached to their outer surfaces (45). The in vitro preparation of the administered blood platelets comprises the steps of: collecting a number of blood platelets (46), either from the same mammal or from an immunologically compatible mammal, using the well known apheresis procedure; and incubating the collected blood platelets in a solution having biotinylated anti-platelet antibodies-avidin or avidin like complexes (47) within it for a time sufficient for the complexes to attach to the outer surface of the platelets (45). The administered platelets will attach to the tumor through the formation of biotin-avidin-biotin or biotin-avidin like-biotin linkages (48) with the biotinylated anti-tumor antibodies already attached to the tumor.

This 2 step method provides means for attaching the blood platelets to the tumor, while avoiding the formation of thrombi within the blood vessels, and thus, it can be used for elderly patients and patients having a tendency to develop infarctions.

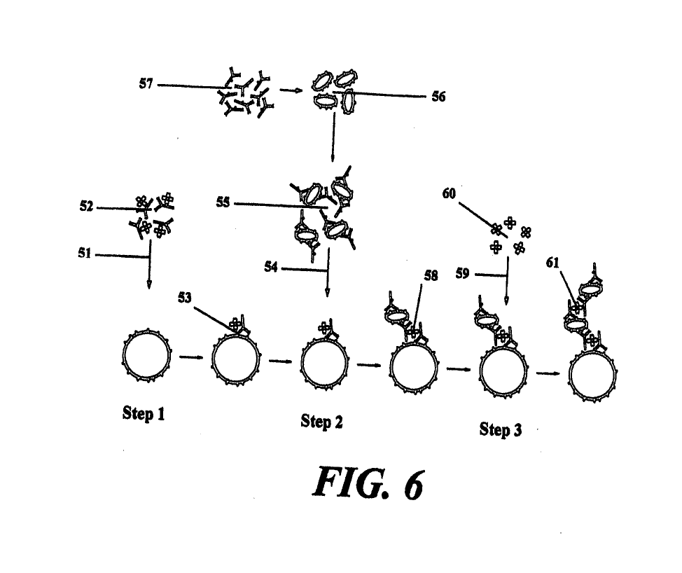

FIG. 6 is a schematic representation of another 3 step method, showing the use of the multicomponent conjugates provided in the present invention for treating a mammal suffering from cancer.

The provided method comprises the steps of:

1) Intravenous injection (51) of a number of biotinylated anti-tumor antibodies-avidin or avidin like complexes (52). The administered complexes will attach themselves to the tumor (53).

2) Intravenous injection (54) of a number of, in vitro prepared, blood platelets having biotinylated anti-platelet antibodies attached to their outer surfaces (55). The in vitro preparation of the administered blood platelets comprises the steps of: collecting a number of blood platelets (56), either from the same mammal or from an immunologically compatible mammal, using the well known apheresis procedure; and incubating the collected blood platelets in a solution having biotinylated anti-platelet antibodies (57) within it for a time sufficient for the antibodies to attach to the outer surface of the platelets. The administered platelets will attach to the tumor cells through the formation of biotin-avidin-biotin or biotin-avidin like-biotin linkages (58) with the complexes already attached to the tumor.

3) Intravenous injection (59) of a number of streptavidin, avidin, or one of their chemically modified molecules (60). The administered avidin or avidin like molecules will attach the extra freely circulating blood platelets having biotinylated antibodies attached to their outer surfaces to the blood platelets already attached to the tumor through biotin-avidin-biotin or biotin-avidin like-biotin linkages (61).

This 3 step method also provides means for accelerating the process of formation of blood clots within the blood vessels around and in-between the cancer cells. However, the administration of the avidin or avidin like molecules (60) in step 3 may be associated with clumping of the freely circulating platelets having biotinylated antibodies attached to their outer surfaces (55), leading to the formation of multiple thrombi within the blood vessels. So, this method is to be used very cautiously in elderly patients and in patients having a tendency to develop infarctions. Optionally, the third step may be omitted, which enables the use of this method in elderly patients and patients having a tendency to develop infarctions.

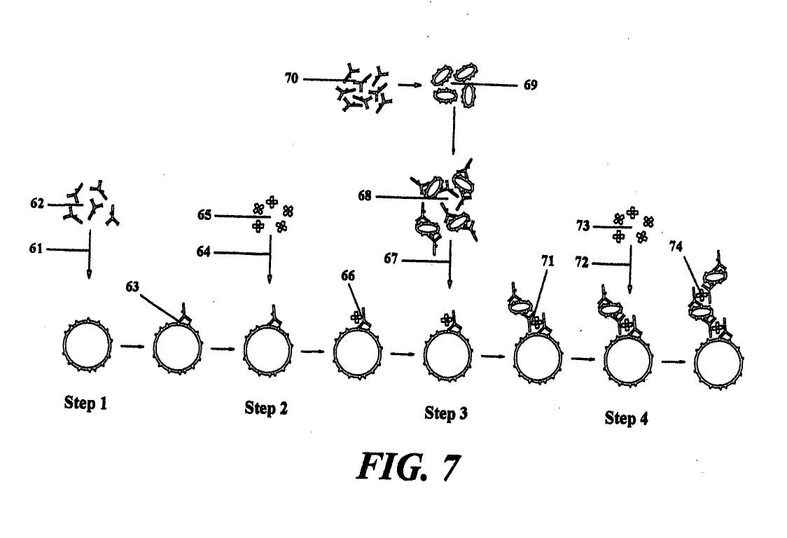

FIG. 7 is a schematic representation of a 4 step method, showing the use of the multicomponent conjugates provided in the present invention for treating a mammal suffering from cancer.

The provided method comprises the steps of:

1) Intravenous injection (61) of a number of biotinylated anti-tumor antibodies (62). The administered anti-tumor antibodies will attach themselves to the tumor (63).

2) Intravenous injection (64) of a number of streptavidin, avidin, or one of their chemically modified molecules (65). The administered avidin or avidin like molecules will attach to the anti-tumor antibodies already attached to the tumor through a biotin-avidin or biotin-avidin like linkages (66).

3) Intravenous injection (67) of a number of, in vitro prepared, blood platelets having biotinylated anti-platelet antibodies attached to their outer surfaces (68). The in vitro preparation of the administered blood platelets comprises the steps of: collecting a number of blood platelets (69), either from the same mammal or from an immunologically compatible mammal, using the well known apheresis procedure; and incubating the collected blood platelets in a solution having biotinylated anti-platelet antibodies (70) within it for a time sufficient for the antibodies to attach to the outer surface of the platelets. The administered platelets will attach to the tumor through the formation of biotin-avidin-biotin or biotin-avidin like-biotin linkages (71) with the conjugates already attached to the tumor.

4) Intravenous injection (72) of a number of streptavidin, avidin, or one of their chemically modified molecules (73). The administered avidin or avidin like molecules will attach the extra freely circulating blood platelets having biotinylated antibodies attached to their outer surfaces to the blood platelets already attached to the tumor through biotin-avidin-biotin or biotin-avidin like-biotin linkages (74).

This 4 step method also provides means for accelerating the process of formation of blood clots within the blood vessels around and in-between the cancer cells. However, the administration of the avidin or avidin like molecules (73) in step 4 may be associated with clumping of the freely circulating platelets having biotinylated antibodies attached to their outer surfaces (68), leading to the formation of multiple thrombi within the blood vessels. So, this method is to be used very cautiously in elderly patients and in patients having a tendency to develop infarctions. Optionally, the fourth step may be omitted, which enables the use of this method in elderly patients and patients having a tendency to develop infarctions.

Certain modifications and improvements will occur to those skilled in the art upon a reading of the foregoing description. All modifications and improvements have been deleted herein for the sake of conciseness and readability but are properly within the scope of the following claims.

1. A multicomponent conjugate comprising a formula A--B--C, wherein “A” comprises at least one specific anti-tumor binding protein recognizing a tumor associated antigen or receptor, “B” comprises at least one molecule to which “A” and “C” bind, and “C” comprises at least one specific anti-platelet binding protein recognizing a receptor or an antigen present on the outer surface of a blood platelet.

2. The multicomponent conjugate of claim 1, wherein “A” is an antibody or a binding fragment of an antibody recognizing an antigen present on the outer surface of a cancer cell.

3. The multicomponent conjugate of claim 1, wherein “A” is an antibody or a binding fragment of an antibody recognizing an antigen present on the outer surface of a component of a tumor associated vasculature or stroma.

4. The multicomponent conjugate of claim 1, wherein “B” comprises a streptavidin or avidin molecule, and from 2 to 4 biotin molecules.

5. The multicomponent conjugate of claim 1, wherein “C” is an antibody or a binding fragment of an antibody recognizing an antigen present on the outer surface of a blood platelet.

6. A multicomponent conjugate comprising a formula A--C, wherein “A” comprises at least one specific anti-tumor binding protein recognizing a tumor associated antigen or receptor, and “C” comprises at least one specific anti-platelet binding protein recognizing a receptor or an antigen present on the outer surface of a blood platelet.

7. The multicomponent conjugate of claim 6, wherein “A” is an antibody or a binding fragment of an antibody recognizing an antigen present on the outer surface of a cancer cell.

8. The multicomponent conjugate of claim 6, wherein “A” is an antibody or a binding fragment of an antibody recognizing an antigen present on the outer surface of a component of a tumor associated vasculature or stroma.

9. The multicomponent conjugate of claim 6, wherein “C” is an antibody or a binding fragment of an antibody recognizing an antigen present on the outer surface of a blood platelet.

10. In a mammal, a method for destroying tumor cells, comprising administering to said mammal a number of multicomponent conjugates, each having the formula A--C, wherein “A” comprises at least one specific anti-tumor binding protein recognizing a tumor associated antigen or receptor, and “C” comprises at least one specific anti-platelet binding protein recognizing a receptor or an antigen present on the outer surface of a blood platelet.

11. The method of claim 10, wherein the said mammal is a human.

12. In a mammal, a method for destroying tumor cells, comprising administering to said mammal a number of multicomponent conjugates, each having the formula A--B--C, wherein “A” comprises at least one specific anti-tumor binding protein recognizing a tumor associated antigen or receptor, “B” comprises at least one molecule to which “A” and “C” bind, and “C” comprises at least one specific anti-platelet binding protein recognizing a receptor or an antigen present on the outer surface of a blood platelet.

13. The method of claim 12, wherein the multicomponent conjugates are prepared in vitro and administered to the said mammal by intravenous injection.

14. The method of claim 12, wherein “A” is an anti-tumor antibody recognizing a tumor associated antigen, “B” comprises an avidin, or an avidin like molecule and 2 biotin molecules, and “C” is an anti-platelet antibody recognizing an antigen present on the outer surface of a blood platelet, with the multicomponent conjugates being assembled in vivo within the body of the said mammal.

15. The method of claim 14, wherein the said in vivo assembling of the multicomponent conjugates within the body of the mammal comprises the steps of:
a) intravenous injection of a number of multicomponent conjugates, each including at least one specific anti-tumor binding protein recognizing a tumor associated antigen or receptor, and at least one specific anti-platelet binding protein recognizing a receptor or an antigen present on the outer surface of a blood platelet;
b) intravenous injection of a number of biotinylated anti-platelet antibodies; and
c) intravenous injection of a number of avidin, or avidin like molecules.

16. The method of claim 14, wherein the said in vivo assembling of the multicomponent conjugates within the body of the mammal comprises the steps of:
a) intravenous injection of a number of biotinylated anti-tumor antibodies; and
b) intravenous injection of a number of biotinylated anti-platelet antibodies-avidin or avidin like complexes.

17. The method of claim 14, wherein the said in vivo assembling of the multicomponent conjugates within the body of the mammal comprises the steps of:
a) intravenous injection of a number of biotinylated anti-tumor antibodies-avidin or avidin like complexes;
b) intravenous injection of a number of biotinylated anti-platelet antibodies; and
c) intravenous injection of a number of avidin or avidin like molecule.

18. The method of claim 14, wherein the said in vivo assembling of the multicomponent conjugates within the body of the mammal comprises the steps of:
a) intravenous injection of a number of biotinylated anti-tumor antibodies and a number of biotinylated anti-platelet antibodies; and
b) intravenous injection of a number of avidin or avidin like molecules.

19. The method of claim 14, wherein the said in vivo assembling of the multicomponent conjugates within the body of the mammal comprises the steps of:
a) Intravenous injection of a number of biotinylated anti-tumor antibodies; and
b) Intravenous injection of a number of, in vitro prepared, blood platelets having biotinylated anti-platelet antibodies-avidin or avidin like complexes attached to their outer surfaces.

20. The method of claim 14, wherein the said in vivo assembling of the multicomponent conjugates within the body of the mammal comprises the steps of:
a) intravenous injection of a number of biotinylated anti-tumor antibodies-avidin or avidin like complexes;
b) intravenous injection of a number of, in vitro prepared, blood platelets having biotinylated anti-platelet antibodies attached to their outer surfaces; and
c) intravenous injection of a number of avidin or avidin like molecules.

21. The method of claim 14, wherein the said in vivo assembling of the multicomponent conjugate within the body of the mammal comprises the steps of:
a) intravenous injection of a number of biotinylated anti-tumor antibodies;
b) intravenous injection of a number of avidin or avidin like molecules;
c) intravenous injection of a number of, in vitro prepared, blood platelets having biotinylated anti-platelet antibodies attached to their outer surfaces; and
d) intravenous injection of a number of avidin or avidin like molecules.

22. The method of claim 14, wherein the said mammal is a human.
